(19) United States
(12) Patent Application Publication
(10) Pub. No.: US 2026/0079119 A1
(43) Pub. Date: Mar. 19, 2026

(54) MONITORING STRUCTURE WITHOUT CHIP

(71) Applicants: INVENTEC (PUDONG) TECHNOLOGY CORPORATION, Shanghai (CN); INVENTEC CORPORATION, Taipei City (TW)

(72) Inventors: Wei-Chen CHENG, Taipei City (TW); Yi-Cheng SHIH, Taipei City (TW); Yu WU, Taipei City (TW); Hsin-Hung LIN, Taipei City (TW); Yu Shu TAI, Taipei City (TW)

(73) Assignees: INVENTEC (PUDONG) TECHNOLOGY CORPORATION, Shanghai (CN); INVENTEC CORPORATION, Taipei City (TW)

(21) Appl. No.: 19/239,361

(22) Filed: Jun. 16, 2025

(30) Foreign Application Priority Data
Sep. 18, 2024 (CN) 202411303261.0

(51) Int. Cl. G01N 22/04; A61B 5/20; A61F 13/42; A61F 13/84; G01N 33/493

(52) U.S. Cl. CPC G01N 22/04; A61B 5/208; A61F 13/42; G01N 33/493; A61F 2013/424; A61F 2013/8488

(57) ABSTRACT

A monitoring structure without chip including at least one first monitoring component. The at least one first monitoring component includes a first substrate and a first signal sensing portion. The first signal sensing portion includes a first signal sensing section, a second signal sensing section, a third signal sensing section and a fourth signal sensing section. The first signal sensing section, the second signal sensing section, the third signal sensing section and the fourth signal sensing section are disposed along a periphery of the substrate. The first signal sensing section and the third signal sensing section are connected to two opposite ends of the second signal sensing section, respectively. The fourth signal sensing section is connected to the third signal sensing section, and is spaced apart from the first signal sensing section via a notch.

### CROSS-REFERENCE TO RELATED APPLICATIONS

This non-provisional application claims priority under 35 U.S. C. § 119(a) on Patent Application No(s). 202411303261.0 filed in China, on Sep. 18, 2024, the entire contents of which are hereby incorporated by reference.

### BACKGROUND OF THE INVENTION

### Technical Field of the Invention

The invention relates to a monitoring structure without chip, more particularly to a monitoring structure without chip including at least one signal sensing portion and a notch.

### Description of the Related Art

Currently, RFID (Radio Frequency Identification) technology has been widely applied. For example, the RFID technology can be applied in fields such as signal monitoring, cargo tracking, internal asset tracking within enterprises, smart inventory management, contactless payment, quality management for production and healthcare. The RFID technology is typically implemented in a form of tags attached to objects that are being monitored or managed.

Generally, the RFID tags are composed of antennas and chips. However, a manufacturing and installation cost of the chips is high. In addition, a large amount of water during a manufacturing process of the chips is consumed, and a large amount of carbon dioxide and various heavy metals such as arsenic, lead, or cadmium can be emitted, thereby causing environmental degradation. Therefore, how to reduce the manufacturing cost of the tags and reduce emission of harmful substances during the manufacturing process of the tags is one of the key issues that researchers need to address.

### SUMMARY OF THE INVENTION

The invention provides a monitoring structure without chip for reducing a manufacturing cost of the monitoring structure and reducing emission of harmful substances during a manufacturing process of the monitoring structure.

One embodiment of the invention provides a monitoring structure without chip including at least one first monitoring component. The at least one first monitoring component includes a first substrate and a first signal sensing portion. The first signal sensing portion includes a first signal sensing section, a second signal sensing section, a third signal sensing section and a fourth signal sensing section. The first signal sensing section, the second signal sensing section, the third signal sensing section and the fourth signal sensing section are disposed along a periphery of the substrate. The first signal sensing section and the third signal sensing section are connected to two opposite ends of the second signal sensing section, respectively. The fourth signal sensing section is connected to the third signal sensing section, and is spaced apart from the first signal sensing section via a notch.

According to the monitoring structure without chip disclosed in the above embodiment, the monitoring structure without chip includes the first monitoring component. The fourth signal sensing section of the first monitoring component is spaced apart from the first signal sensing section via a notch. Accordingly, a frequency response can be generated via the aforementioned configuration. Therefore, a functionality for monitoring can be achieved without chips based on the frequency response, and the manufacturing cost of the monitoring structure can be reduced. In addition, the emission of harmful substances during the manufacturing process of the monitoring structure without chip can be reduced.

### BRIEF DESCRIPTION OF THE DRAWINGS

The invention will become more fully understood from the detailed description given herein below and the accompanying drawings which are given by way of illustration only and thus are not limitative of the invention and wherein:

FIG. 1 is a perspective view of a first monitoring component of a monitoring structure without chip in accordance with a first embodiment of the invention;

FIG. 2 is a plane view of the first monitoring component of the monitoring structure without chip in FIG. 1;

FIG. 3 is a graph showing a frequency response of the first monitoring component of the monitoring structure without chip in FIG. 1;

FIG. 4 is a plane view of a plurality of first monitoring components of a monitoring structure without chip in accordance with a second embodiment of the invention;

FIG. 5 is a graph showing a frequency response of the plurality of first monitoring components of the monitoring structure without chip in FIG. 4;

FIG. 6 is a perspective view of one of a plurality of second monitoring components of a monitoring structure without chip in accordance with a third embodiment of the invention;

FIG. 7 is a plane view of a plurality of first monitoring components and the plurality of second monitoring components of the monitoring structure without chip in accordance with a third embodiment of the invention;

FIG. 8 is a graph showing a frequency response of the plurality of first monitoring components and the plurality of second monitoring components of the monitoring structure without chip in FIG. 7;

FIG. 9 is a plane view of a plurality of first monitoring components and a second monitoring component of a monitoring structure without chip in accordance with a fourth embodiment of the invention;

FIG. 10 is a graph showing a frequency response of the plurality of first monitoring components and the second monitoring component of the monitoring structure without chip in FIG. 9;

FIG. 11 is a plane view of a plurality of first monitoring components and a plurality of second monitoring components of a monitoring structure without chip in accordance with a fifth embodiment of the invention;

FIG. 12 is a graph showing a frequency response of the plurality of first monitoring components and the plurality of second monitoring components of the monitoring structure without chip in FIG. 11;

FIG. 13 is a schematic view showing the usage of the monitoring structure without chip in accordance with the first embodiment to the fifth embodiment of the invention; and

FIG. 14 is another schematic view showing the usage of the monitoring structure without chip in accordance with the first embodiment to the fifth embodiment of the invention.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

In addition, the terms used in the invention, such as technical and scientific terms, have its own meanings and can be comprehended by those skilled in the art, unless the terms are additionally defined in the invention. That is, the terms used in the following paragraphs should be read on the meaning commonly used in the related fields and will not be overly explained, unless the terms have a specific meaning in the invention.

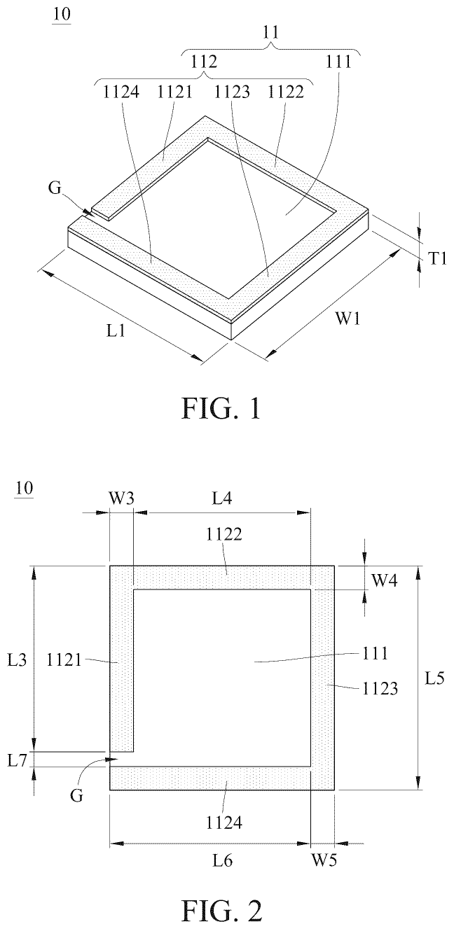

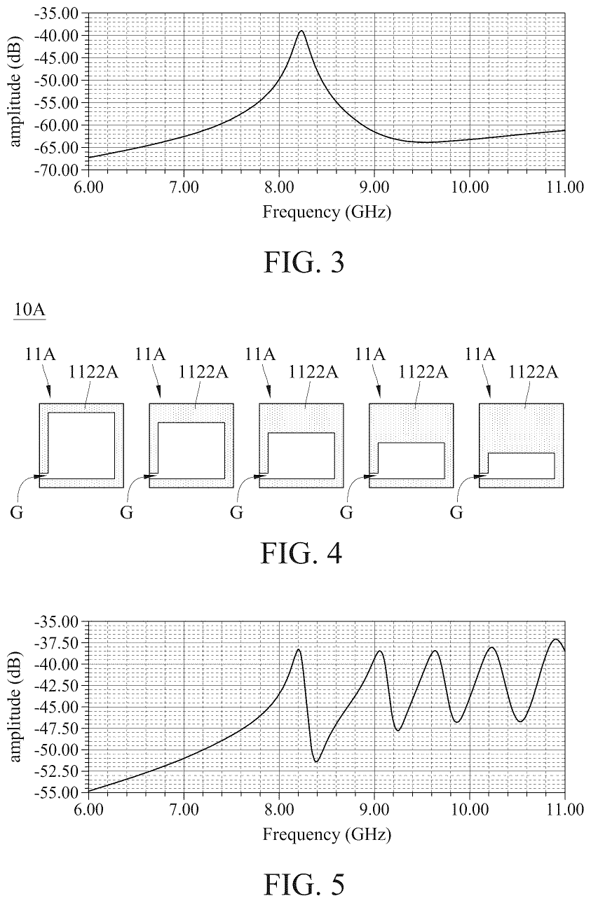

Please refer to FIG. 1 to FIG. 3, where FIG. 1 is a perspective view of a first monitoring component 11 of a monitoring structure without chip 10 in accordance with a first embodiment of the invention, FIG. 2 is a plane view of the first monitoring component 11 of the monitoring structure without chip 10 in FIG. 1, and FIG. 3 is a graph showing a frequency response of the first monitoring component 11 of the monitoring structure without chip 10 in FIG. 1.

In this embodiment, the monitoring structure without chip 10 is configured to generate an echo signal (i.e., frequency response) at a specific frequency when receiving a radar plane wave, so as to monitor an object to be monitored (not shown). The monitoring structure without chip 10 includes a first monitoring component 11. The first monitoring component 11 includes a first substrate 111 and a first signal sensing portion 112. The first substrate 111 is, for example, rectangular. In addition, the first substrate 111 is, for example, a dielectric substrate, such as Rogers RT6035 HTC board, and the first signal sensing portion 112 is, for example, a conductive metal material such as copper. For example, the first signal sensing portion 112 is made of copper foil. It should be noted that a thickness of the first signal sensing portion 112 in FIG. 1 is exaggerated for distinguishing the first signal sensing portion 112 from the first substrate 111 and allows a thickness of the first substrate 111 to be recognized. In practice, the thickness of the first signal sensing portion 112 is extremely thin enough to be negligible.

The first signal sensing portion 112 includes a first signal sensing section 1121, a second signal sensing section 1122, a third signal sensing section 1123 and a fourth signal sensing section 1124. The first signal sensing section 1121, the second signal sensing section 1122, the third signal sensing section 1123 and the fourth signal sensing section 1124 are disposed along a periphery of the first substrate 111. The first signal sensing section 1121 and the third signal sensing section 1123 are connected to two opposite ends of the second signal sensing section 1122, respectively. The fourth signal sensing section 1124 is connected to the third signal sensing section 1123, and is spaced apart from the first signal sensing section 1121 via a notch G. The notch G is located away from corners of the first substrate 111, and the notch G and the first signal sensing section 1121 are located on the same side edge of the first substrate 111.

In this embodiment, as shown in FIG. 3, an amplitude of a background signal is, for example, −65 dB, while the first monitoring component 11 of the monitoring structure without chip 10 generates, for example, the frequency response of −39 dB at the frequency of 8.2 GHz. That is, the monitoring structure without chip 10 can generate, for example, an echo signal of 26 dB.

In this embodiment, there is one first monitoring component 11 merely, but the invention is not limited thereto. In other embodiments, please refer to FIG. 1 to FIG. 5, where FIG. 4 is a plane view of a plurality of first monitoring components 11A of a monitoring structure without chip 10A in accordance with a second embodiment of the invention, and FIG. 5 is a graph showing a frequency response of the plurality of first monitoring components 11A of the monitoring structure without chip 10A in FIG. 4. The monitoring structure without chip 10A of this embodiment is similar to the monitoring structure without chip 10 of the first embodiment, and the main difference between them will be described below, and the same parts between them can be referred to the aforementioned paragraphs with the reference to FIG. 1 to FIG. 3 and will not be repeatedly introduced hereinafter.

In this embodiment, there are multiple first monitoring components 11A. The first monitoring components 11A are arranged, for example, along a straight line. Widths of the second signal sensing sections 1122A of the first monitoring components 11A are different from each other, and are less than or equal to lengths of the first signal sensing sections 1121. That is, the second signal sensing sections 1122A of the first monitoring components 11A are spaced apart from the notches G, and do not cover the notches G.

In detail, the widths of the second signal sensing sections 1122A of the first monitoring components 11A are gradually increased from one of the first monitoring components 11A located on one end of the straight line to another first monitoring component 11A located on another end of the straight line. That is, the second signal sensing sections 1122A taper towards the first monitoring components 11A located on the one end of the straight line. The degrees by which the second signal sensing sections 1122A of any two adjacent first monitoring components 11A taper are identical. However, the invention is not limited thereto, as long as all of the second signal sensing sections 1122A are spaced apart from the notches G, and do not cover the notches G, the degrees by which the second signal sensing sections 1122A of any two adjacent first monitoring components 11A taper may be different from each other.

In this embodiment, the wider the widths of the second signal sensing sections 1122A, the higher the frequency of the frequency response generated by the first monitoring components 11A of the monitoring structure without chip 10A. For example, there are five first monitoring components 11A, and, as shown in FIG. 5, the five first monitoring components 11A generate the frequency responses of, for example, −38 dB, −38 dB, −38 dB, −38 dB and −37 dB at the frequencies of 8.2 GHz, 9.1 GHz, 9.6 GHz, 10.2 GHz and 10.9 GHz, respectively.

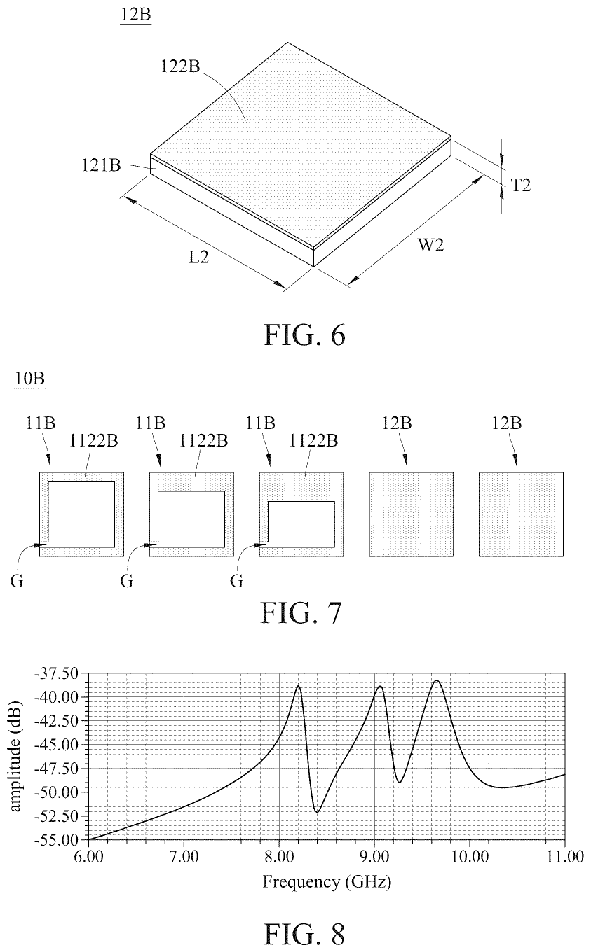

Please refer to FIG. 1 to FIG. 8, where FIG. 6 is a perspective view of one of a plurality of second monitoring components 12B of a monitoring structure without chip 10B accordance with a third embodiment of the invention, FIG. 7 is a plane view of a plurality of first monitoring components 11B and the plurality of second monitoring components 12B of the monitoring structure without chip 10B in accordance with a third embodiment of the invention, and FIG. 8 is a graph showing a frequency response of the plurality of first monitoring components 11B and the plurality of second monitoring components 12B of the monitoring structure without chip 10B in FIG. 7. The monitoring structure without chip 10B of this embodiment is similar to the monitoring structure without chip 10A of the second embodiment, and the main difference between them will be described below, and the same parts between them can be referred to the aforementioned paragraphs with the reference to FIG. 1 to FIG. 5 and will not be repeatedly introduced hereinafter.

In this embodiment, the monitoring structure without chip 10B may include a plurality of second monitoring components 12B. Each of the second monitoring components 12B includes a second substrate 121B and a second signal sensing portion 122B. The second signal sensing portion 122B covers a side of the second substrate 121B. The second substrate 121B is, for example, a dielectric substrate, such as Rogers RT6035 HTC board, and the second signal sensing portion 122B is, for example, a conductive metal material such as copper. For example, the second signal sensing portion 122B is made of copper foil. It should be noted that a thickness of the second signal sensing portion 122B in FIG. 6 is exaggerated for distinguishing the second signal sensing portion 122B from the second substrate 121B and allows a thickness of the second substrate 121B to be recognized. In practice, the thickness of the second signal sensing portion 122B is extremely thin enough to be negligible. The second monitoring components 12B and the first monitoring components 11B are arranged, for example, along a straight line. The second monitoring components 12B do not generate an echo signal when receiving the radar plane wave.

In this embodiment, for example, there are three first monitoring components 11B, and there are two second monitoring components 12B. The two second monitoring components 12B are located on, for example, a side (e.g. right side) of the three first monitoring components 11B. As shown in FIG. 8, the three first monitoring components 11B generate the frequency responses of, for example, −39 dB, −39 dB and −38 dB at the frequencies of 8.2 GHz, 9.1 GHz and 9.7 GHz, respectively. In addition, the second monitoring components 12B do not generate the frequency responses.

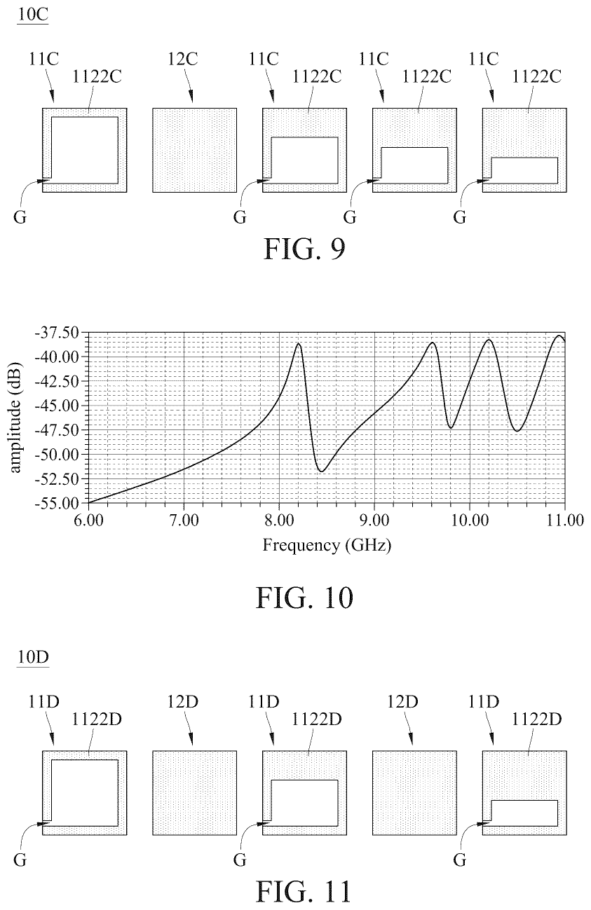

Please refer to FIG. 1 to FIG. 10, where FIG. 9 is a plane view of a plurality of first monitoring components 11C and a second monitoring component 12C of a monitoring structure without chip 10C in accordance with a fourth embodiment of the invention, and FIG. 10 is a graph showing a frequency response of the plurality of first monitoring components 11C and the second monitoring component 12C of the monitoring structure without chip 10C in FIG. 9. The monitoring structure without chip 10C of this embodiment is similar to the monitoring structure without chip 10A of the second embodiment, and the main difference between them will be described below, and the same parts between them can be referred to the aforementioned paragraphs with the reference to FIG. 1 to FIG. 8 and will not be repeatedly introduced hereinafter.

In this embodiment, the second monitoring component 12C and the first monitoring components 11C are arranged, for example, along a straight line. For example, there are four first monitoring components 11C, and there is one second monitoring component 12C merely. One of the four first monitoring components 11C and the other first monitoring components 11C are located on two opposite sides of the second monitoring component 12C, respectively. As shown in FIG. 10, the four first monitoring components 11C generate the frequency responses of, for example, −39 dB, −39 dB, −38 dB and −38 dB at the frequencies of 8.2 GHz, 9.6 GHz, 10.2 GHz and 10.9 GHz, respectively. In addition, the second monitoring component 12C does not generate the frequency response.

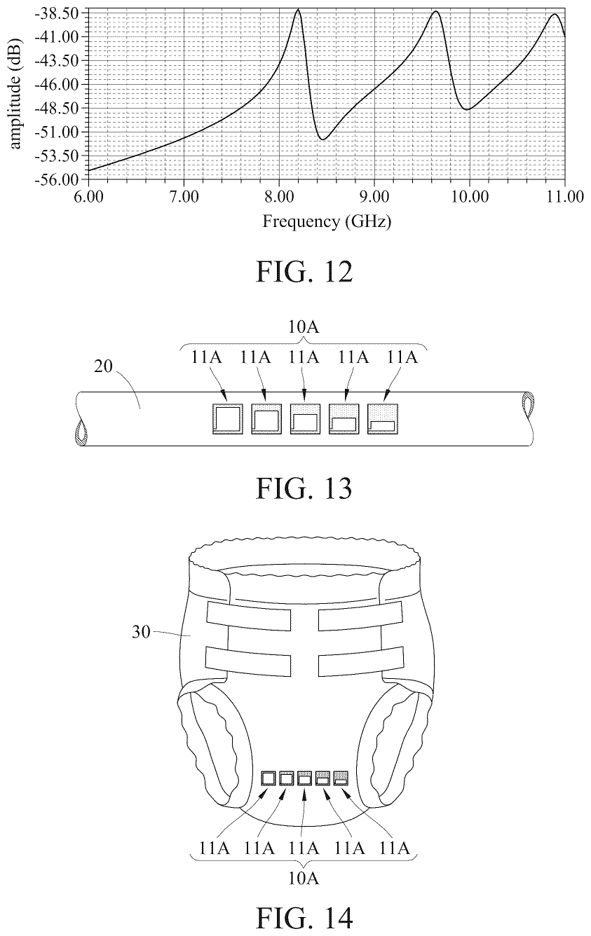

Please refer to FIG. 11 to FIG. 12, where FIG. 11 is a plane view of a plurality of first monitoring components 11D and a plurality of second monitoring components 12D of a monitoring structure without chip 10D in accordance with a fifth embodiment of the invention, and FIG. 12 is a graph showing a frequency response of the plurality of first monitoring components 11D and the plurality of second monitoring components 12D of the monitoring structure without chip 10D in FIG. 11. The monitoring structure without chip 10D of this embodiment is similar to the monitoring structure without chip 10A of the second embodiment, and the main difference between them will be described below, and the same parts between them can be referred to the aforementioned paragraphs with the reference to FIG. 1 to FIG. 10 and will not be repeatedly introduced hereinafter.

In this embodiment, the second monitoring components 12D and the first monitoring components 11D are arranged, for example, along a straight line. For example, there are four three monitoring components 11D, and there are two second monitoring component 12D. The first monitoring components 11D and the second monitoring components 12D are arranged, for example, alternately. As shown in FIG. 12, the three first monitoring components 11D generate the frequency responses of, for example, −38 dB, −38 dB and −39 dB at the frequencies of 8.2 GHz, 9.6 GHz and 10.9 GHz, respectively. In addition, the second monitoring components 12D does not generate the frequency response.

Please refer to FIG. 1 to FIG. 13, where FIG. 13 is a schematic view showing the usage of the monitoring structure without chip (e.g. the monitoring structure without chip 10A of the second embodiment of the invention) in accordance with the first embodiment to the fifth embodiment of the invention. The monitoring structure without chip 10A is disposed on, for example, a urinary catheter 20.

When a user urinates into the urinary catheter 20 and a urine flows through the first monitoring components 11A of the monitoring structure without chip 10A, a signal attenuation occurs sequentially on these first monitoring components 11A. By dividing a distance between two adjacent first monitoring components 11A by the time difference between the occurrences of the signal attenuations of any two adjacent first monitoring components 11A, the flow rate of the urine can be obtained. In addition, by multiplying the flow rate of the urine per unit time by an inner diameter of the urinary catheter 20, a urine flow volume per unit time can be obtained. Accordingly, a urination condition of the user can be monitored.

Please refer to FIG. 1 to FIG. 14, where FIG. 14 is another schematic view showing the usage of the monitoring structure without chip (e.g. the monitoring structure without chip 10A of the second embodiment of the invention) in accordance with the first embodiment to the fifth embodiment of the invention. The monitoring structure without chip 10A is disposed on, for example, a diaper 30.

When the monitoring structure without chip 10A is disposed on a front side of the diaper 30, the monitoring structure without chip 10A can be configured to monitor a urination volume of a user. In detail, a urine has a high dielectric constant (e.g., greater than or equal to 79 and less than or equal to 81). Therefore, when the user urinates onto the diaper 30 and the urine flows through the first monitoring components 11A of the monitoring structure without chip 10A, an overall dielectric constant of the first monitoring components 11A of the monitoring structure without chip 10A can be increased, such that a frequency response generated by the first monitoring components 11A can be shifted to a frequency band with lower frequency range and a signal attenuation occurs on the first monitoring components 11A. Accordingly, the urination volume of the user can be estimated according to a degree of the shift in the frequency response and a degree of the signal amplitude attenuation, thereby determining whether the diaper 30 is required to be replaced.

Similarly, when the monitoring structure without chip 10A is disposed on a rear side of the diaper 30 and the user defecates onto the diaper 30, since the feces contacting with the first monitoring components 11A of the monitoring structure without chip 10A can increase the overall dielectric constant of the first monitoring components 11A of the monitoring structure without chip 10A, the monitoring structure without chip 10A can be configured to monitor a defecation volume of the user to determine whether the diaper 30 is required to be replaced.

In this embodiment, the monitoring structure without chip 10/10A includes the first monitoring component(s) 11/11A, or the monitoring structure without chip 10B/10C/10D includes the first monitoring components 11B/11C/11D and the second monitoring component(s) 12B/12C/12D. The fourth signal sensing section(s) 1124 of the first monitoring component(s) 11/11A/11B/11C/11D is spaced apart from the first signal sensing section(s) 1121 via a notch(es) G. Accordingly, the frequency response(s) can be generated via the aforementioned configuration. Therefore, a functionality for monitoring can be achieved without chips based on the frequency response(s), and a manufacturing cost of a monitoring structure can be reduced. For example, the manufacturing cost of the monitoring structure without chip 10/10A/10B/10C/10D may be reduced to less than 1/20 of the manufacturing cost of a conventional monitoring structure including one or more chips. In addition, since no chips are required in the monitoring structure without chip 10/10A/10B/10C/10D, the emission of harmful substances during a manufacturing process of the monitoring structure without chip 10/10A/10B/10C/10D can be reduced.

In the above embodiments, a length L1 of the first substrate(s) 111, a width W1 of the first substrate(s) 111, a length L2 of the second substrate(s) 121B and a width W2 of the second substrate(s) 121B are, for example, 3.8 millimeters. A thickness T1 of the first substrate(s) 111 and a thickness T2 of the second substrate(s) 121B are, for example, 0.762 millimeters.

In the above embodiments, a length L3 of the first signal sensing section(s) 1121 is, for example, 3.15 millimeters. A width W3 of the first signal sensing section(s) 1121 is, for example, 0.4 millimeters. Lengths L4 of the second signal sensing sections 1122 and 1122A-1122D are, for example, 3 millimeters. Widths W4 of the second signal sensing sections 1122 and 1122A-1122D are, for example, less than or equal to 0.4 millimeters. A length L5 of the third signal sensing section(s) 1123 is, for example, 3.8 millimeters. A width W5 of the third signal sensing section(s) 1123 is, for example, 0.4 millimeters. A length L6 of the fourth signal sensing section(s) 1124 is, for example, 3.4 millimeters. A width W6 of the fourth signal sensing section(s) 1124 is, for example, 0.4 millimeters. In addition, a length L7 of the notch(es) G is, for example, less than or equal to 0.25 millimeters.

In the above embodiments, the widths of the second signal sensing sections 1122A-1122D of the first monitoring components 11A-11D are different, but the invention is not limited thereto. In other embodiments, the widths of the second signal sensing sections of the first monitoring components may be identical, while widths of the third signal sensing sections of the first monitoring components may be, for example, different. The third signal sensing sections are spaced apart from the first signal sensing sections. That is, the third signal sensing sections of the first monitoring components do not cover the notches.

In the above embodiments, the first monitoring components 11A are arranged along a straight line, the first monitoring components 11B and the second monitoring components 12B are arranged along a straight line, the first monitoring components 11C and the second monitoring components 12C are arranged along a straight line, and the first monitoring components 11D and the second monitoring components 12D are arranged along a straight line, but the invention is not limited thereto. In other embodiments, the first monitoring components may be arranged, for example, in an array, and the first monitoring components and the second monitoring components may be arranged, for example, in an array.

According to the monitoring structure without chip disclosed in the above embodiments, the monitoring structure without chip includes the first monitoring component(s), or the monitoring structure without chip includes the first monitoring components and the second monitoring component(s). The fourth signal sensing section(s) of the first monitoring component(s) is spaced apart from the first signal sensing section(s) via a notch(es). Accordingly, the frequency response(s) can be generated via the aforementioned configuration. Therefore, the functionality for monitoring can be achieved without chips based on the frequency response(s), and the manufacturing cost of the monitoring structure can be reduced. In addition, the emission of harmful substances during the manufacturing process of the monitoring structure without chip can be reduced.

It will be apparent to those skilled in the art that various modifications and variations can be made to the invention. It is intended that the specification and examples be considered as exemplary embodiments only, with the scope of the invention being indicated by the following claims.

What is claimed is:

1. A monitoring structure without chip, comprising:
at least one first monitoring component, comprising;
a first substrate; and
a first signal sensing portion, comprising a first signal sensing section, a second signal sensing section, a third signal sensing section and a fourth signal sensing section, wherein the first signal sensing section, the second signal sensing section, the third signal sensing section and the fourth signal sensing section are disposed along a periphery of the first substrate, the first signal sensing section and the third signal sensing section are connected to two opposite ends of the second signal sensing section, respectively, and the fourth signal sensing section is connected to the third signal sensing section, and is spaced apart from the first signal sensing section via a notch.

2. The monitoring structure without chip according to claim 1, wherein the notch is located away from corners of the first substrate, and the notch and the first signal sensing section are located on a side edge of the first substrate.

3. The monitoring structure without chip according to claim 2, wherein the at least one first monitoring component comprises a plurality of first monitoring components, the plurality of first monitoring components are arranged along a straight line or in an array, widths of the second signal sensing sections of the plurality of first monitoring components are different from each other, and the widths of the second signal sensing sections of the plurality of first monitoring components are less than or equal to lengths of the first signal sensing sections.

4. The monitoring structure without chip according to claim 3, wherein the plurality of first monitoring components are arranged along a straight line, and the widths of the second signal sensing sections of the plurality of first monitoring components are gradually increased from one of the plurality of first monitoring components located on one end of the straight line to another one of the plurality of first monitoring components located on another end of the straight line.

5. The monitoring structure without chip according to claim 4, wherein the second signal sensing sections taper towards the at least one first monitoring component located on the one end of a straight line.

6. The monitoring structure without chip according to claim 3, further comprising at least one second monitoring component, wherein the at least one second monitoring component and the plurality of first monitoring components are arranged along a straight line or in an array, the at least one second monitoring component comprises a second substrate and a second signal sensing portion, and the second signal sensing portion covers a side of the second substrate.

7. The monitoring structure without chip according to claim 6, wherein a length of the first substrate, a width of the first substrate, a length of the second substrate and a width of the second substrate are 3.8 millimeters, and a thickness of the first substrate and a thickness of the second substrate are 0.762 millimeters.

8. The monitoring structure without chip according to claim 2, wherein the at least one first monitoring component comprises a plurality of first monitoring components, the plurality of first monitoring components are arranged along a straight line or in an array, widths of the third signal sensing sections of the plurality of first monitoring components are different, and the third signal sensing sections are spaced apart from the first signal sensing sections.

9. The monitoring structure without chip according to claim 1, wherein a length of the first signal sensing section is 3.15 millimeters, a width of the first signal sensing section is 0.4 millimeters, a length of the second signal sensing section is 3 millimeters, a width of the second signal sensing section is less than or equal to 0.4 millimeters, a length of the third signal sensing section is 3.8 millimeters, a width of the third signal sensing section is 0.4 millimeters, a length of the fourth signal sensing section is 3.4 millimeters, and a width of the fourth signal sensing section is 0.4 millimeters.

10. The monitoring structure without chip according to claim 1, wherein a length of the notch is less than or equal to 0.25 millimeters.
